(19) United States
(12) Patent Application Publication
(10) Pub. No.: US 2014/0371605 A1
(43) Pub. Date: Dec. 18, 2014

(54) APPARATUS AND METHOD FOR MONITORING SITUATIONS DANGEROUS TO THE HUMAN BODY

(71) Applicants: We Duke Cho, Gyeonggi-do (KR); Tae Yun Kim, Gyeonggi-do (KR); Hui Jung Park, Gyeonggi-do (KR); Jin Hyung Kim, Gyeonggi-do (KR); Yang Weon Kim, Incheon (KR)

(72) Inventors: We Duke Cho, Gyeonggi-do (KR); Tae Yun Kim, Gyeonggi-do (KR); Hui Jung Park, Gyeonggi-do (KR); Jin Hyung Kim, Gyeonggi-do (KR); Yang Weon Kim, Incheon (KR)

(73) Assignee: Ajou University Industry Cooperation Foundation, Gyeonggi-do (KR)

(21) Appl. No.: 14/352,383

(22) PCT Filed: Oct. 18, 2012
(86) PCT No.: PCT/KR2012/008519

§ 371 Date: Apr. 17, 2014

(30) Foreign Application Priority Data
Oct. 18, 2011 (KR) 10-2011-0106536

(51) Int. Cl. A61B 5/00; A61B 5/0205

(52) U.S. Cl. CPC A61B 5/4818; A61B 5/0205; A61B 5/7282; A61B 5/7225; A61B 5/08
USPC 600/484

(57) ABSTRACT

Provided are an apparatus and a method for monitoring a dangerous situation of a human body. The dangerous situation such as the apnea or suffocation of the human body is determined based on variations in one of a cardiac impulse amplitude signal and a respiration amplitude signal of the human body and is transmitted to a wired or wireless terminal, thereby monitoring the dangerous situation of the human body in a sleep state.

### FIELD

The present disclosure relates to an apparatus and a method for monitoring a dangerous situation of a human body, and more particularly, to an apparatus and a method for monitoring a dangerous situation of a human body, capable of alerting or monitoring dangerous situations such as the suffocation or apnea of the human body.

### BACKGROUND

A death from suffocation designates a death caused by suffocation.

The death from suffocation indicates a death caused by a disorder occurring in gas exchange performed by respiration, that is, inhaling oxygen necessary for a body and exhaling carbon dioxide.

There may be a lot of causes of the death from suffocation. For example, there is suffocation caused by smoke generated from fire or a child suffocation caused by an airbag of a vehicle, which inflates in a car accident.

Particularly, in the case of infants, there have been reported deaths from suffocation caused by a comforter in a sleep state or sleeping on his or her stomach.

While an infant is turning over or is lying with his or her face down, his or her face is covered with bedding such as covers and a blanket and has breathing difficulties.

For example, in the case of putting an infant to sleep on a bed or a mattress filled with soft cotton, when the infant does not move for several or several ten minutes while the infant is turning over or lying on his or her face, a face of the infant is covered by the mattress in such a way that the infant may suffer from breathing difficulties and a death from suffocation may occur.

This may occur to infants, patients with a cardiac disorder, or the old.

Infant apnea indicates a state, in which an infant does not take a breath for 20 seconds or more.

The infant apnea designates a pathological apnea state, in which a 37 week-old infant or older breathes heavily or does not take a breath for 20 seconds or more. Symptoms herein include a low pulse rate, cyanoderma, and a pale face, which reaches approximately a suffocation state.

Also, an elastic degree of muscles rapidly decreases. Although a causal relationship is not clearly unraveled, this may be a cause of sudden infant death syndromes.

Accordingly, an apparatus for preventing and monitoring a dangerous situation caused by one of suffocation and apnea of infants or the old is necessary.

This simply detects a dangerous situation and gives an alarm to allow a guardian to quickly recognize the situation.

### SUMMARY

Embodiments provide an apparatus and a method for monitoring a dangerous situation of a human body in a sleep state by determining the dangerous situation such as apnea and suffocation of the human body based on variations in cardiac impulse or a respiration amplitude signal and transmitting a result thereof to a wireless or wired terminal.

According to an aspect of the present invention, an apparatus for monitoring a dangerous situation of a human body includes a detection unit extracting one of a cardiac impulse amplitude signal and a respiration amplitude signal of the human body from a biosignal in a first state of the human body and a biosignal in a second state of the human body and a dangerous situation determination unit determining whether the human body is in a dangerous situation or not, based on variations in one of the cardiac impulse amplitude signal and the respiration amplitude signal when it changes from the first state to the second state.

The biosignals in the first state and the second state may be obtained by a piezo sensor for sensing variations in one of cardiac impulse and respiration of the human body.

The apparatus may further include an analog-digital (A/D) converter converting a signal obtained by the piezo sensor into a digital signal and a band pass filter allowing a reference frequency component of the digital signal. Herein the biosignals in the first state and the second state may be obtained through a process, in which signals obtained by the piezo sensor are converted into digital signals by the A/D converter and biosignals are obtained after the band pass filter filters the digital signals.

The apparatus may further include an output unit transmitting an alert for a dangerous situation to a wired or wireless terminal when the dangerous situation determination unit determines the dangerous situation.

One of the cardiac impulse amplitude signal and the respiration amplitude signal of the human body may be a digital signal having a certain level within a certain time interval.

The first state may indicate a state, in which the human body lies on a bed, and the second state may indicate a state, in which the human body lies on a face thereof.

The band pass filter may be one of a high band pass filter removing a low band, which removes a respiration signal of the human body and allows only a cardiac impulse signal of the human body to pass, and a low band pass filter removing a high band, which removes the cardiac impulse signal of the human body and allows only the respiration signal of the human body to pass.

A plurality of first amplitude signals of the cardiac impulse signal may be detected based on a difference between a peak signal connecting maximum values of the cardiac impulse and a valley signal connecting minimum values of the cardiac impulse of an output signal of the band pass filter, and the cardiac impulse amplitude signal may be detected by extracting a maximum first amplitude signal within a reference time interval.

A plurality of first amplitude signals of the respiration signal may be detected based on a difference between a peak signal connecting maximum values of the respiration and a valley signal connecting minimum values of the respiration of an output signal of the band pass filter, and the respiration amplitude signal may be detected by extracting a maximum first amplitude signal within a reference time interval.

The dangerous situation determination unit may determine a situation to be dangerous when variations in the cardiac impulse amplitude signal increases more than a reference value when it changes from the first state to the second state.

The dangerous situation determination unit may determine a situation to be dangerous when variations in the respiration amplitude signal are reduced more than a reference value when it changes from the first state to the second state.

Phases of the peak signal and the valley signal may be allowed to be identical to each other.

According to another aspect of the present invention, a method of monitoring a dangerous situation of a human body includes detecting one of a cardiac impulse amplitude signal and a respiration amplitude signal of the human body from a biosignal in a first state and a biosignal in a second state of the human body and determining whether the human body is in the dangerous situation, based on variations in one of the cardiac impulse amplitude signal and the respiration amplitude signal when it changes from the first state to the second state.

The biosignals in the first state and the second state may be obtained by a piezo sensor sensing variations in one of cardiac impulse and respiration of the human body.

The method may further include converting a signal obtained by the piezo sensor into a digital signal and allowing a reference frequency component of the digital signal to pass, wherein the biosignals in the first state and the second state are biosignals obtained through the converting the signals obtained by the piezo sensor into the digital signal and then the allowing of the reference frequency component of the digital signal to pass.

The method may further include transmitting an alert of the dangerous situation from a dangerous situation determination unit to a wired or wireless terminal.

One of the cardiac impulse amplitude signal and the respiration amplitude signal may be a digital signal having a certain level within a certain time interval.

The first state may indicate a state, in which the human body lies on back thereof on a bed, and the second state may indicate a state, in which the human body lies on face thereof.

The allowing of the reference frequency component of the digital signal to pass may be one of allowing only a cardiac impulse signal of the human body to pass while removing a respiration signal of the human body and allowing only a respiration signal of the human body to pass while removing the cardiac impulse signal.

A plurality of first amplitude signals of the cardiac impulse signal may be detected based on a difference between a peak signal connecting maximum values of the cardiac impulse and a valley signal connecting minimum values of the cardiac impulse of an output signal of the allowing of the reference frequency component of the digital signal to pass, and the cardiac impulse amplitude signal may be detected by extracting a maximum first amplitude signal within a reference time interval.

A plurality of first amplitude signals of the respiration signal may be detected based on a difference between a peak signal connecting maximum values of the respiration and a valley signal connecting minimum values of the respiration of an output signal of the allowing of the reference frequency component of the digital signal to pass, and the respiration amplitude signal may be detected by extracting a maximum first amplitude signal within a reference time interval.

The determining whether the human body is in the dangerous situation may be determining a situation to be dangerous when variations in the cardiac impulse amplitude signal increases more than a reference value when it changes from the first state to the second state.

The determining whether the human body is in the dangerous situation may be determining a situation to be dangerous when variations in the respiration amplitude signal is reduced more than a reference value when it changes from the first state to the second state.

The method may further include allowing phases of the peak signal and the valley signal to be identical.

### Advantageous Effect

According to the embodiments, the apparatus and method for monitoring a dangerous situation of a human body may prevent a death of the human body by monitoring the dangerous situation of the human body through determining the dangerous situation such as apnea and suffocation of the human body based on variations in cardiac impulse or a respiration amplitude signal and transmitting a result thereof to a wired or wireless terminal.

### DRAWINGS

The above and other features and advantages of the present invention will become more apparent by describing in detail exemplary embodiments thereof with reference to the attached drawings in which:

FIG. 1 is a configuration view of an apparatus for monitoring a dangerous situation of a human body according to an exemplary embodiment;

FIG. 2 is a view illustrating an example of using an apparatus for monitoring a dangerous situation of a human body according to another exemplary embodiment;

FIG. 3 illustrates examples of a first state biosignal and a second state biosignal of a detection unit according to another exemplary embodiment;

FIG. 4 illustrates examples of cardiac impulse amplitude signals extracted from the first state biosignal and the second state biosignal of the detection unit, shown in FIG. 3;

FIG. 5 illustrates an example of determining a dangerous situation of a human body based on variations in a cardiac impulse amplitude signal of a dangerous situation determination unit according to another exemplary embodiment;

FIG. 6 illustrates a cardiac impulse signal and a respiration signal included in one of the first state biosignal and the second state biosignal;

FIG. 7 illustrates an example of a peak signal and a valley signal of cardiac impulse according to an exemplary embodiment;

FIG. 8 illustrates an example of an amplitude variation signal of the cardiac impulse;

FIG. 9 illustrates an example of a peak signal in the amplitude variation signal of the cardiac impulse;

FIG. 10 illustrates an example of a final amplitude signal of the cardiac impulse extracted from one of the first state biosignal and the second state biosignal;

FIG. 11 is a flowchart illustrating a method of monitoring a dangerous situation of a human body according to an exemplary embodiment; and

FIG. 12 is a flowchart illustrating a process of extracting one of the cardiac impulse amplitude signal and the respiration amplitude signal from one of the first situation body signal and the second situation body signal.

### DETAILED DESCRIPTION

To fully understand advantages in operation and objects achieved by embodiments of the present invention, it is necessary to refer to the attached drawings illustrating exemplary embodiments of the present invention and contents disclosed in the drawings.

Hereinafter, the exemplary embodiments of the present invention will be described in detail with reference to the attached drawings. In the drawings, like reference numerals refer to like elements.

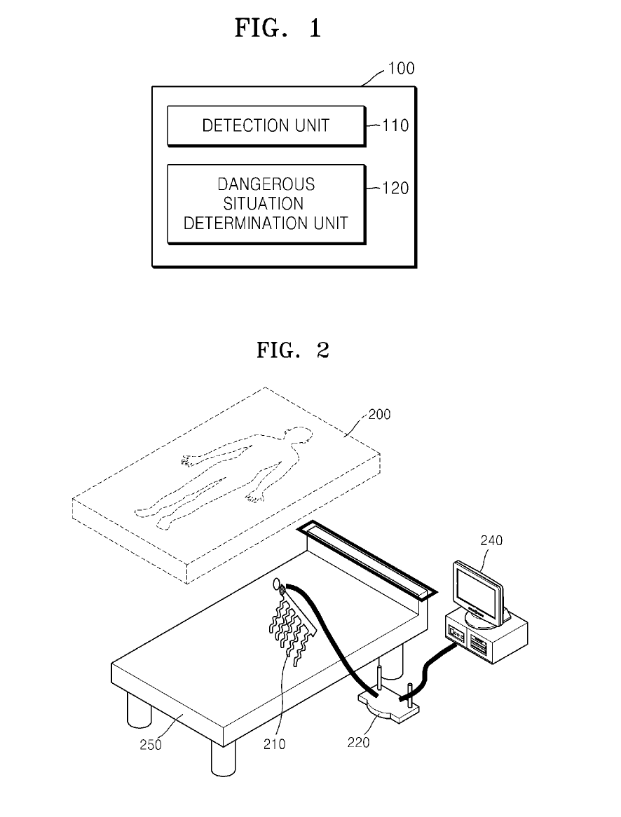

FIG. 1 is a configuration view of an apparatus 100 for monitoring a dangerous situation of a human body according to an exemplary embodiment.

The apparatus 100 includes a detection unit 110 and a dangerous situation determination unit 120.

The detection unit 110 receives a body signal of a human body in a first state and a body signal of the human body in a second state, obtained by a sensor.

The biosignals of the human body may be obtained by a piezo sensor.

The biosignals in the first state and the second state received at the detection unit 110 are obtained by converting signals obtained by the piezo-sensor into digital signals by an analog-digital (A/D) converter.

A band pass filter may be further included to receive biosignals having a reference frequency component of the digital signals.

The band pass filter, for example, may be a high band pass filter removing a low band, which removes a respiration signal of a human body and only allows a cardiac impulse signal of the human body to pass, or may be a low band pass filter removing a high band, which removes the cardiac impulse signal of the human body and only allows the respiration signal of the human body.

As an example of the high band pass filter removing the low band, which removes the respiration signal of the human body and only allows the cardiac impulse signal of the human body, there is a finite impulse response (FIR) filter.

As an example of the low band pass filter removing the high band, which removes the cardiac impulse signal of the human body and only allows the respiration signal of the human body, there is an infinite impulse response (IIR) filter.

The detection unit 110 extracts amplitude signals of cardiac impulse and respiration of the human body from the inputted biosignals.

The biosignals of the first state and the second state include a cardiac impulse signal and a respiration signal of the human body, as shown in FIG. 6.

The dangerous situation determination unit 120 determines whether the human body is in a dangerous situation based on variations of one of the cardiac impulse signal and the respiration amplitude signal when the human body changes from the first state to the second state.

The first state may be a state, in which the human body, for example, an infant lies on a bed. The second state may be a state, in which the human body lies on his or her face.

An infant suffocation may occur while an infant is lying on his or her face. The apparatus and method for monitoring the dangerous situation of the human body apply a phenomenon, in which the amplitude of a cardiac impulse signal more greatly increases in a case of lying on his or her face than in a case of lying on his or her back.

Infant apnea designates a state, in which an infant does not take a breath for a certain time. It is applied that the amplitude of respiration is greatly reduced in an apnea state, that is, the second state, than the first state of normally taking a breath.

The apparatus and the method are provided to monitor and determine a dangerous situation and may further include an output unit (not shown) for informing a guardian of the dangerous situation.

Various methods of informing the guardian of the dangerous situation in the output unit are performed through a wired or wireless terminal.

For example, a notification may be transmitted through a wired warning light or a message on a wireless terminal.

FIG. 2 is a view illustrating an example of using an apparatus 240 for monitoring a dangerous situation of a human body.

FIG. 2 illustrates an example of using the apparatus 240 when an infant lies on a bed.

The apparatus 240 receives a biosignal obtained by a piezo-sensor 210 integrated to a bed 250 and converted by an A/D converter 220 into a digital signal.

A band pass filter for allowing a reference frequency component of the digital signal to pass may be further included.

The piezo-sensor 210 obtains and transmits a biosignal of a first state, in which a human body 200 lies on a bed 250, and a biosignal of a second state, in which the human body 200 lies on his or her face, respectively.

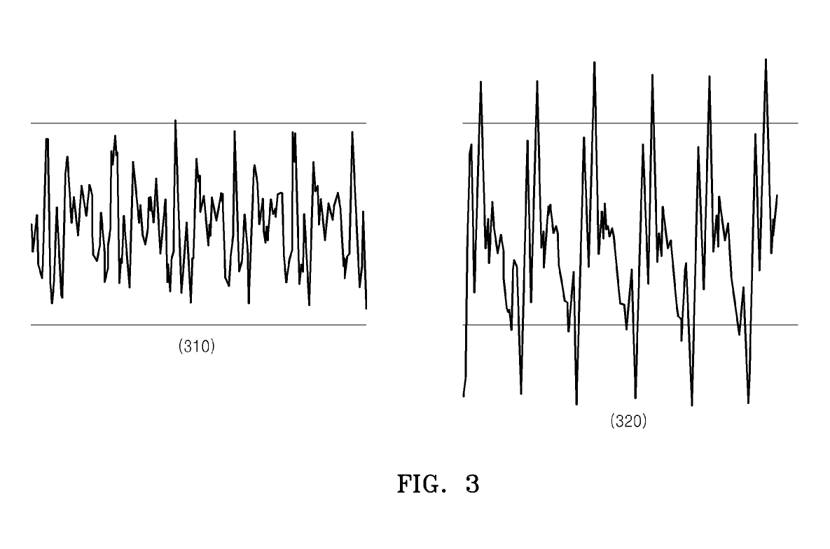

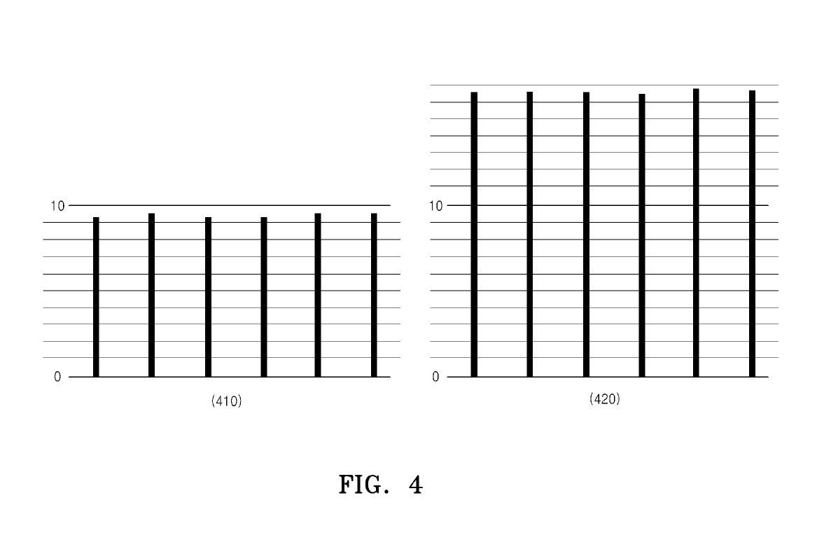

A detection unit of the apparatus 240 extracts one of a cardiac impulse signal and a respiration amplitude signal shown in FIG. 4 from biosignals shown in FIG. 3.

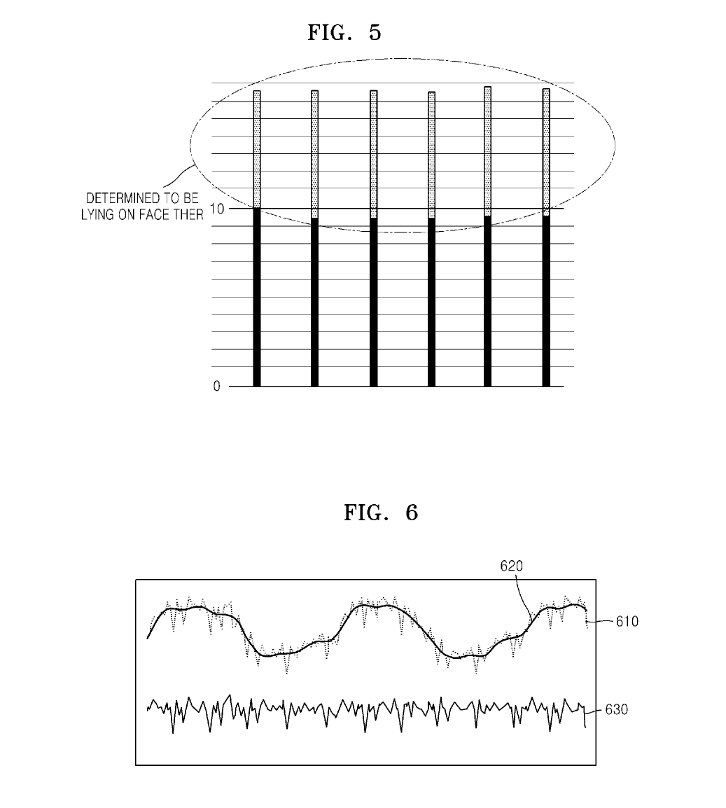

A dangerous situation determination unit of the apparatus 240 determines whether the human body 200 is in a dangerous situation, based on variations shown in FIG. 5 of the cardiac impulse signal or the respiration amplitude signal shown in FIG. 4.

Since the infant is lying on his or her face on the bed or is in an apnea state, it is determined as a dangerous situation, which needs to notify a guardian of a danger of a death.

FIG. 3 illustrates examples of a first state biosignal and a second state biosignal of the detection unit.

In FIG. 3, when an infant lies on a bed, a first state biosignal 310 indicates a biosignal when the infant lies on his or her back on the bed and a second state biosignal 320 indicates a biosignal when the infant lies on his or her face on the bed.

FIG. 4 illustrates examples of cardiac impulse amplitude signals extracted from the first state biosignal and the second state biosignal of the detection unit.

Amplitude signals of cardiac impulse and respiration of a human body, which are output signals of the detection unit, are digital signals having a certain level for each certain time interval.

In FIG. 4, there are shown examples of amplitude signals with respect to cardiac impulse in one of the first state biosignal and the second state biosignal inputted to the detection unit of the apparatus 240.

The amplitude of a cardiac impulse amplitude signal 410 when the infant lies on his or her back on the bed is smaller than the amplitude of a cardiac impulse amplitude signal 420 when the infant lies on his or her face on the bed.

FIG. 5 illustrates an example of determining a dangerous situation of a human body based on variations in a cardiac impulse amplitude signal of the dangerous situation determination unit.

The dangerous situation determination unit of the apparatus 240 determines that the infant lies on his or her face on the bed from a result of FIG. 4, in which the amplitude of the cardiac impulse amplitude signal 420 when the infant lies on his or her face on the bed more greatly increases than the amplitude of the cardiac impulse amplitude signal 410 when the infant lies on his or her back on the bed, and transmits a determination to a wired or wireless terminal of a guardian through the output unit.

In a dangerous situation of apnea, the amplitude is more greatly reduced than a normal respiration state.

Hereinafter, with reference to FIGS. 6 to 10, a process of extracting an amplitude signal of cardiac impulse from a biosignal obtained by the piezo-sensor 210 in the detection unit of the apparatus 240 will be described in detail.

FIG. 6 illustrates a cardiac impulse signal and a respiration signal included in one of the first state biosignal and the second state biosignal;

In FIG. 6, a signal obtained by the piezo-sensor as a biosignal inputted to the detection unit of the apparatus 240 is converted into a digital signal by the A/D converter 220.

A signal only having a reference frequency component is received through a band pass filter only allowing the reference frequency component to pass from the digital signal.

As an example of a high band pass filter removing a low band, which removes a respiration signal of a human body and only allows a cardiac impulse signal of the human body, there is an FIR filter.

As an example of a low band pass filter removing a high band, which removes the cardiac impulse signal of the human body and only allows the respiration signal of the human body, there is an IIR filter.

In FIG. 6, a biosignal 610 inputted to the detection unit includes a respiration signal 620 and a cardiac impulse signal 630.

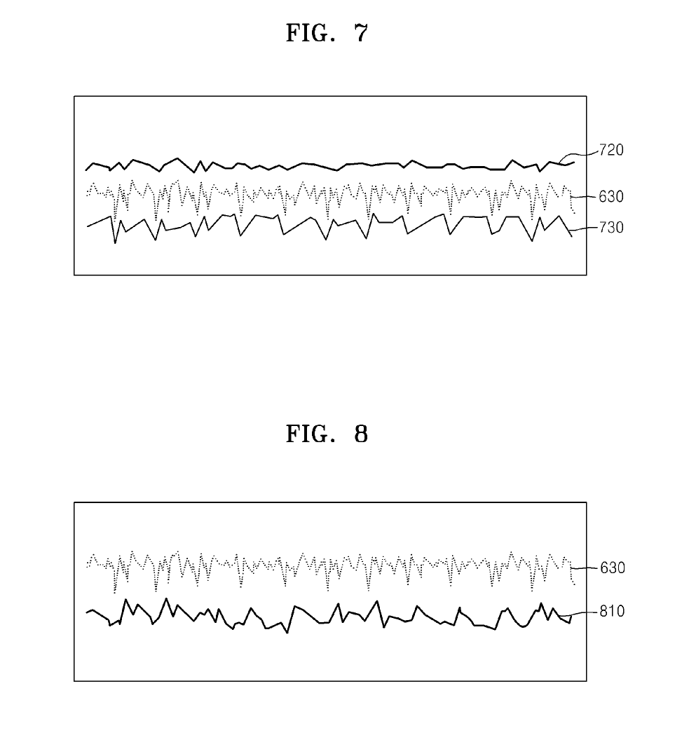

FIG. 7 illustrates an example of a peak signal and a valley signal of cardiac impulse.

The peak signal 720 is connecting maximum values of the cardiac impulse in biosignals inputted to the detection unit, and the valley signal 730 is connecting minimum values of the cardiac impulse.

To allow properties of the peak signal 720 to be identical to properties of the valley signal 730, an operation of shifting a phase may be further included.

When a difference between the peak signal 720 connecting the maximum values and the valley signal 730 connecting the minimum values is obtained, a great value is extracted from a section greatly varying in amplitude and a small value is extracted from a section less varying in amplitude.

FIG. 8 illustrates an example of an amplitude variation signal of the cardiac impulse.

As the difference between the peak signal 720 and the valley signal 730, an amplitude variation signal 810 is obtained.

The amplitude variation signal 810 indicating variations in amplitude is obtained from a biosignal of cardiac impulse.

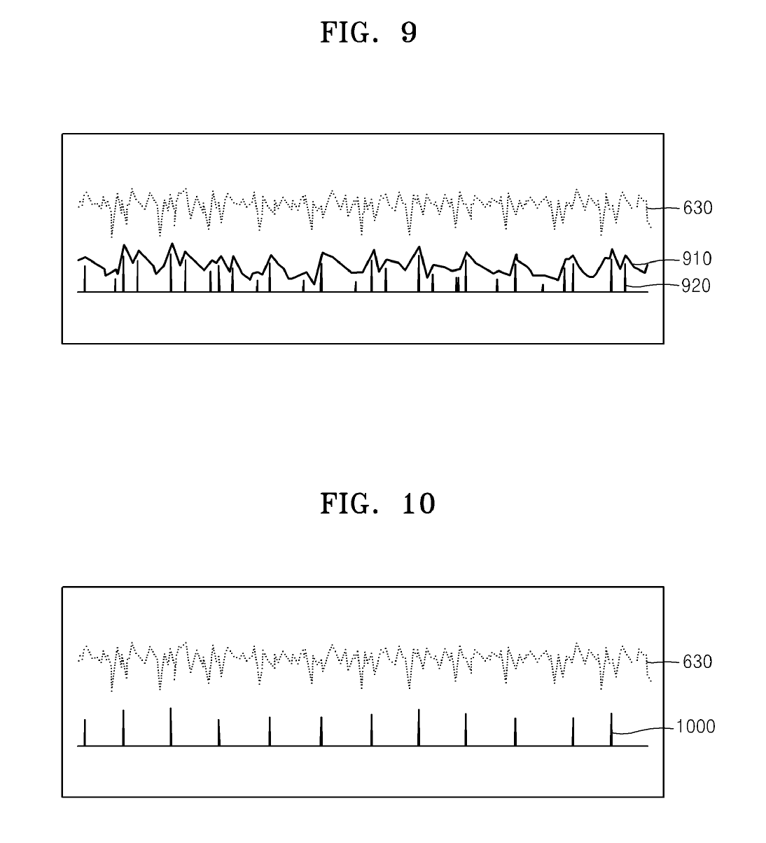

FIG. 9 illustrates an example of a peak signal in an amplitude variation signal of cardiac impulse.

A great value is extracted from a section of an amplitude variation signal 910, greatly varying in amplitude. A small value is extracted from a second less varying in amplitude.

A plurality of preliminary amplitude signals 920 of cardiac impulse are obtained by extracting only peak values from the amplitude variation signal 910.

FIG. 10 illustrates an example of a final amplitude signal of the cardiac impulse, extracted from one of the first state body signal and the second state body signal.

Not all the preliminary amplitude signals 920 obtained in FIG. 9 may be the final amplitude signal of the cardiac impulse.

A noise component periodically occurring may be present or the cardiac impulse itself may be detected with multi-peaks.

Accordingly, it is necessary to periodically detect a maximum peak point.

Within a reference time interval, for example, 0.4 seconds, small peak points are ignored and the maximum peak value is extracted and detected as a final amplitude signal 1000 of the cardiac impulse.

The amplitude signal 1000 of the cardiac impulse obtained in FIG. 10 becomes one of the cardiac impulse amplitude signal 410 and the cardiac impulse amplitude signal 420.

In the case of an amplitude signal of respiration, a peak value is extracted from a respiration biosignal passing through an IIR filter, which is a low band pass filter removing a high band, removing a cardiac impulse signal of a human body and allowing only a respiration signal of the human body to pass. The peak value is not extracted in the case of apnea.

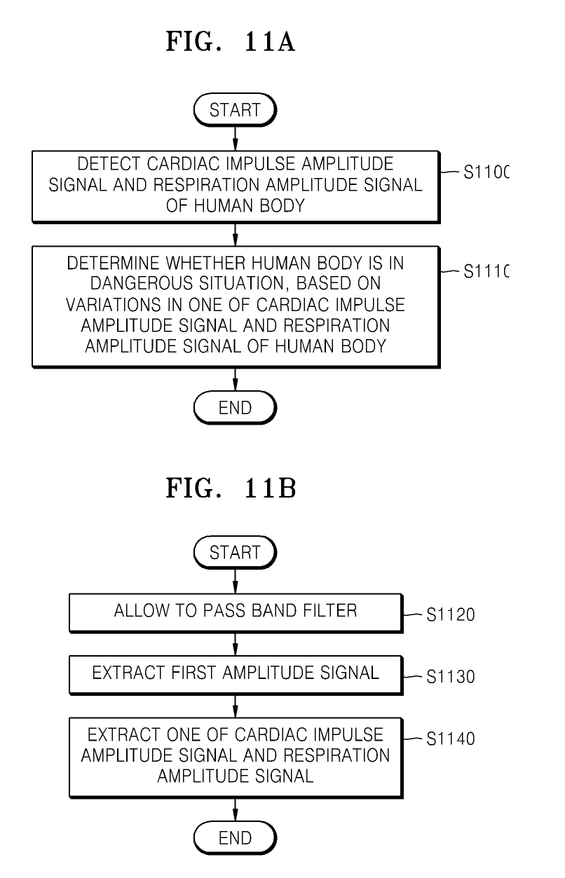

FIG. 11 is a flowchart illustrating a method of monitoring a dangerous situation of a human body according to an exemplary embodiment.

A detection unit of an apparatus for monitoring a dangerous situation of a human body extracts amplitude signals of cardiac impulse and respiration of the human body from a biosignal in a first state of the human body and a biosignal in a second state of the human body (S1100).

The biosignals in the first state and the second state received by the detection unit are obtained by converting signals obtained by a piezo-sensor into digital signals by an A/D converter.

One of an FIR filter and an IIR filter may be further included to receive a biosignal having a certain frequency band from the digital signal.

That is, to extract only a cardiac impulse signal from the biosignal, an FIR filter is used, which is a high band pass filter removing a low band. To extract only a respiration signal, an IIR filter is used, which is a low band pass filter removing a high band.

The biosignals in the first state and the second state are shown in FIG. 3 and include a cardiac impulse signal and a respiration signal as shown in FIG. 6, respectively.

The cardiac impulse signal of the human body obtained by the detection unit becomes a signal 1000 displaying maximum peak values in one of FIGS. 4 and 10.

A dangerous situation determination unit of the apparatus determines whether the human body is in a dangerous situation, based on variations of one of the cardiac impulse signal and the respiration amplitude signal when the human body changes from the first state to the second state.

It is applied to the apparatus and the method for monitoring the dangerous situation of the human body that the amplitude of one of a cardiac impulse signal and a respiration signal when the human body lies on his or her face more greatly increases than the amplitude thereof when the human body lies.

Infant apnea designates a state, in which an infant does not take a breath for a certain time. It is applied that the amplitude of respiration is greatly reduced in an apnea state, that is, the second state, than the first state of normally taking a breath.

That is, based on variations in a cardiac impulse signal or a respiration amplitude signal, a dangerous situation of the infant, that is, the human body is determined and an alert is sent to a guardian.

FIG. 12 is a flowchart illustrating a process of extracting one of the cardiac impulse amplitude signal and the respiration amplitude signal from one of the first state biosignal and the second state biosignal.

A biosignal inputted to the diction unit of the apparatus is obtained by converting a raw biosignal obtained by a piezo sensor into a digital signal by an A/D converter.

A band pass filter may be further included and a signal having a certain frequency band may be received (S1120).

To remove a respiration signal of a human body and to allow only a cardiac impulse signal of the human body, as an example of a high band pass filter removing a low band, an FIR filter may be used.

To remove a cardiac impulse signal of a human body and to allow only a respiration signal of the human body, as an example of a low band pass filter removing a high band, an IIR filter may be used

Based on a difference between a peak signal connecting maximum values of one of cardiac impulse and respiration and a valley signal connecting minimum values of one of the cardiac impulse and respiration, a plurality of first amplitude signals of one of a cardiac impulse signal and a respiration signal (S1130).

A final cardiac impulse amplitude signal is detected by extracting a maximum first amplitude signal within a reference time interval (S1140).

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An apparatus for monitoring a dangerous situation of a human body, comprising:
a detection unit extracting one of a cardiac impulse amplitude signal and a respiration amplitude signal of the human body from a biosignal in a first state of the human body and a biosignal in a second state of the human body; and
a dangerous situation determination unit determining whether the human body is in a dangerous situation or not, based on variations in one of the cardiac impulse amplitude signal and the respiration amplitude signal when it changes from the first state to the second state.

2. The apparatus of claim 1, wherein the biosignals in the first state and the second state are obtained by a piezo sensor for sensing variations in one of cardiac impulse and respiration of the human body.

3. The apparatus of claim 2, further comprising:
an analog-digital (A/D) converter converting a signal obtained by the piezo sensor into a digital signal; and
a band pass filter allowing a reference frequency component of the digital signal,
wherein the biosignals in the first state and the second state are obtained through a process, in which signals obtained by the piezo sensor are converted into digital signals by the A/D converter and biosignals are obtained after the band pass filter filters the digital signals.

4. The apparatus of claim 1, further comprising an output unit transmitting an alert for a dangerous situation to a wired or wireless terminal when the dangerous situation determination unit determines the dangerous situation.

5. The apparatus of claim 1, wherein one of the cardiac impulse amplitude signal and the respiration amplitude signal of the human body is a digital signal having a certain level within a certain time interval.

6. The apparatus of claim 1, wherein the first state indicates a state, in which the human body lies on a bed, and
wherein the second state indicates a state, in which the human body lies on a face thereof.

7. The apparatus of claim 3, wherein the band pass filter is one of a high band pass filter removing a low band, which removes a respiration signal of the human body and allows only a cardiac impulse signal of the human body to pass, and a low band pass filter removing a high band, which removes the cardiac impulse signal of the human body and allows only the respiration signal of the human body to pass.

8. The apparatus of claim 7, wherein a plurality of first amplitude signals of the cardiac impulse signal are detected based on a difference between a peak signal connecting maximum values of the cardiac impulse and a valley signal connecting minimum values of the cardiac impulse of an output signal of the band pass filter, and
wherein the cardiac impulse amplitude signal is detected by extracting a maximum first amplitude signal within a reference time interval.

9. The apparatus of claim 7, wherein a plurality of first amplitude signals of the respiration signal are detected based on a difference between a peak signal connecting maximum values of the respiration and a valley signal connecting minimum values of the respiration of an output signal of the band pass filter, and
wherein the respiration amplitude signal is detected by extracting a maximum first amplitude signal within a reference time interval.

10. The apparatus of claim 1, wherein the dangerous situation determination unit determines a situation to be dangerous when variations in the cardiac impulse amplitude signal increases more than a reference value when it changes from the first state to the second state.

11. The apparatus of claim 1, wherein the dangerous situation determination unit determines a situation to be dangerous when variations in the respiration amplitude signal is reduced more than a reference value when it changes from the first state to the second state.

12. The apparatus according to any one of claims 8 and 9, further comprising wherein phases of the peak signal and the valley signal are allowed to be identical to each other.

13. A method of monitoring a dangerous situation of a human body, comprising:
detecting one of a cardiac impulse amplitude signal and a respiration amplitude signal of the human body from a biosignal in a first state and a biosignal in a second state of the human body; and
determining whether the human body is in the dangerous situation, based on variations in one of the cardiac impulse amplitude signal and the respiration amplitude signal when it changes from the first state to the second state.

14. The method of claim 13, wherein the biosignals in the first state and the second state are obtained by a piezo sensor sensing variations in one of cardiac impulse and respiration of the human body.

15. The method of claim 14, further comprising:
converting a signal obtained by the piezo sensor into a digital signal; and
allowing a reference frequency component of the digital signal to pass,
wherein the biosignals in the first state and the second state are biosignals obtained through the converting the signals obtained by the piezo sensor into the digital signal and then the allowing of the reference frequency component of the digital signal to pass.

16. The method of claim 13, further comprising transmitting an alert of the dangerous situation from a dangerous situation determination unit to a wired or wireless terminal.

17. The method of claim 13, wherein one of the cardiac impulse amplitude signal and the respiration amplitude signal is a digital signal having a certain level within a certain time interval.

18. The method of claim 13, wherein the first state indicates a state, in which the human body lies on back thereof on a bed, and
wherein the second state indicates a state, in which the human body lies on face thereof.

19. The method of claim 15, wherein the allowing of the reference frequency component of the digital signal to pass is one of allowing only a cardiac impulse signal of the human body to pass while removing a respiration signal of the human body and allowing only a respiration signal of the human body to pass while removing the cardiac impulse signal.

20. The method of claim 19, wherein a plurality of first amplitude signals of the cardiac impulse signal are detected based on a difference between a peak signal connecting maximum values of the cardiac impulse and a valley signal connecting minimum values of the cardiac impulse of an output signal of the allowing of the reference frequency component of the digital signal to pass, and
wherein the cardiac impulse amplitude signal is detected by extracting a maximum first amplitude signal within a reference time interval.

21. The method of claim 19, wherein a plurality of first amplitude signals of the respiration signal are detected based on a difference between a peak signal connecting maximum values of the respiration and a valley signal connecting minimum values of the respiration of an output signal of the allowing of the reference frequency component of the digital signal to pass, and
wherein the respiration amplitude signal is detected by extracting a maximum first amplitude signal within a reference time interval.

22. The method of claim 13, wherein the determining whether the human body is in the dangerous situation is determining a situation to be dangerous when variations in the cardiac impulse amplitude signal increases more than a reference value when it changes from the first state to the second state.

23. The method of claim 13, wherein the determining whether the human body is in the dangerous situation is determining a situation to be dangerous when variations in the respiration amplitude signal is reduced more than a reference value when it changes from the first state to the second state.

24. The method according to any one of claims 20 and 21, further comprising allowing phases of the peak signal and the valley signal to be identical.
